(19) United States
(12) Patent Application Publication
(10) Pub. No.: US 2009/0081797 A1
(43) Pub. Date: Mar. 26, 2009

(54) REACTIVE SURFACE ON A POLYMERIC SUBSTRATE

(76) Inventors: ANDREI G FADEEV, Elmira, NY (US); Santona Pal, Painted Post, NY (US); David Michael Weber, Corning, NY (US)

Correspondence Address: CORNING INCORPORATED, SP-TI-3-1, CORNING, NY, 14831, US

(21) Appl. No.: 12/201,029

(22) Filed: Aug. 29, 2008

Related U.S. Application Data
(60) Provisional application No. 60/967,230, filed on Aug. 31, 2007.

(51) Int. Cl. G01N 33/68; C08J 3/28; C08F 8/32

(52) U.S. Cl. 436/86; 522/87; 522/88; 522/160; 525/54.1; 525/54.2; 525/333.6

(57) ABSTRACT

Plasma treated cyclic polyolefin copolymer surfaces having enhanced binding density for binding biologically active agents and cells are provided. These plasma treated cyclic polyolefin copolymer surfaces may be further enhanced for binding biologically active agents or cells by the application of conjugates. Methods of making and characterizing treated polymer surfaces are also provided.

### CROSS REFERENCE TO RELATED APPLICATIONS

This Application claims the benefit of U.S. Provisional Application Ser. No. 60/967,230 filed Aug. 31, 2007 and entitled “Reactive Surface on a Polymeric Substrate” which is incorporated by reference herein.

### BACKGROUND

The present invention relates generally to the field of polymeric substrates and more specifically to modified polymer substrates that have activated moieties at the surface for binding biologically active compounds. Methods for analyzing and using the treated polymer surfaces are also provided by the present invention.

Polymer substrates that are stiff and strong, easy to melt, extrude and thermoform, optically transparent, chemically inert, resistant to temperature fluctuations and harsh pH conditions, biocompatible and provide excellent moisture barrier properties are desirable in many industries and applications. In the fields of biotechnology, pharmaceutical sciences, food science, cell science, fermentation, and other fields, the interaction between biologically relevant organic compounds and the materials that make up containers or surfaces which come into contact with the biologically relevant compounds are important. In some industries, it is important that organic compounds do not stick to containers. In some applications, it is important that organic compounds stick to surfaces or containers, even in the face of fluid flow, freeze-thaw cycles or other harsh conditions.

It is desirable to develop polymer substrates that exhibit a wide range of attributes amenable to uses in the fields of cell science, pharmaceutical science, biotechnology, fermentation and other fields.

### SUMMARY OF INVENTION

Embodiments of the present invention provide methods for making polymer substrates having a working surface upon which biologically active compounds or cells can bind comprising (1) providing a cyclic polyolefin copolymer; (2) treating the cyclic polyolefin copolymer with plasma to provide functional groups on a surface of the polymer substrate; (3) exposing the polymer surface to a conjugate so that the functional groups on the surface of the polymer substrate form covalent bonds with the conjugate; and, (4) exposing the polymer surface covalently bound to the conjugate to a biologically active compound so that the biologically active compound becomes immobilized on the polymer surface. Further embodiments provide the method where the conjugate 1-ethyl-3-(3-dimethylaminopropyl)carbodiimide (EDC) or a combination of EDC and N-hydroxysuccinimide (NHS), where the biologically active agent may be peptides, proteins, carbohydrates, nucleic acids, lipids, polysaccarides, glycosaminoglycans, proteoglycans, extracellular matrix molecules, cell adhesion molecules, or cells or combinations or fragments thereof. Additional embodiments of the present invention provide polymers made by the disclosed methods. In additional embodiments, the cyclic polyolefin copolymer may be treated with microwave plasma.

In additional embodiments, the polymer substrate has a peptide binding density greater than 0.5 pmol/mm² or greater than 1.5 pmol/mm². In still further embodiments, the present invention provides polymer substrates which provide at least a portion of a flask, a dish, a flat plate, a well plate, a sheet, a bottle, a roller bottle, a container, a pipette, a pipette tip, a tube, a bead, a medical device, a filter device, a film, a membrane, a slide, or a bead.

In further embodiments, the present invention provides a method for making a polymer substrate having a working surface upon which cells can bind including: (1) providing a cyclic polyolefin copolymer; (2) treating the cyclic polyolefin copolymer with plasma to provide functional groups on a surface of the polymer substrate; (3) exposing the polymer surface to a conjugate so that the functional groups on the surface of the polymer substrate form covalent bonds with the conjugate; (4) exposing the polymer surface with covalently bound conjugate to a cell-binding peptide so that the cell binding peptide becomes immobilized on the polymer surface. In additional embodiments, the cell binding peptide may be KGGNGEPRGDTYRAY (SEQ ID NO 3), NGEPRGDTYRAY (SEQ ID NO 4), KGGPQVTRGDVFTMP (SEQ ID NO 5), or a combination of these. In embodiments, the cell binding peptide may be immobilized to the surface via a linker which may be PEG or PEO.

In embodiments, the present invention also provides methods for assessing the number of accessible functional groups on the surface of a polymer available for binding including: (1) providing a plasma-treated cyclic polyolefin copolymer surface having a number of functional groups on its surface to be determined; (2) exposing the plasma-treated cyclic polyolefin copolymer surface to 1-ethyl-3-(3-dimethylaminopropyl)carbodiimide (EDC) and N-hydroxysuccinimide (NHS); (3) exposing the plasma-treated cyclic polyolefin copolymer surface to labeled biological agent or a mixture or labeled and unlabeled biological agents so that biological agent is immobilized to the surface, and; (4) determining the amount of label indirectly bound to the surface.

In still further embodiments, the present invention provides methods for assigning a rating to a polymer surface including the steps of providing a treated polymer surface having a number of functional groups on its surface to be determined; exposing the polymer surface to conjugate so that the functional groups on the surface of the polymer form covalent bonds with the conjugate; exposing the polymer surface covalently bound to the conjugate to labeled biological agent of a mixture or labeled and unlabeled biological agents so that the labeled biological agent becomes immobilized to the polymer surface; determining the amount of label immobilized to the polymer surface; and, assigning a rating to the polymer surface based on the amount of label immobilized to the surface.

In still further embodiments, the present invention provides polymer substrates having a working surface upon which cells can bind comprising a cyclic polyolefin copolymer substrate having plasma-treatment-induced functional groups conjugated to cell-binding peptides.

### BRIEF DESCRIPTION OF THE DRAWINGS

FIG. 1 is a schematic illustration of a polymer with functional groups at or near its surface.

FIG. 2 is a schematic illustration of a polymer with functional groups at its surface.

FIG. 3 is a schematic drawing of biomolecule binding to a polymer surface that has been treated to have functional groups at its surface.

FIG. 4 illustrates chemical schemes for producing a polymer surface which is treated to bind with biologically active compounds having primary amine groups.

FIG. 5 is a fluorescence scan of a calibration curve, comparing peptide conjugated, blocked and untreated wells of a treated surface.

FIGS. 6A and 6B illustrate the results of the fluorescence scans of calibration, conjugation, blocking and background results as measured from a plate as shown in FIG. 5.

FIG. 7 is a graph showing levels of binding of biologically active compounds on three different polymer surfaces.

FIG. 8 is a graph showing HT-1080 cell binding on various surfaces treated according to embodiments of the present invention.

### DETAILED DESCRIPTION

Embodiments of the present invention include plastic surfaces which are treated to improve surface characteristics for applications in industries such as biotechnology, pharmaceutical sciences, food science, cell science, fermentation, bio-sciences and other fields. In embodiments, the present invention provides surfaces, including cyclic polyolefin copolymer surfaces, which have been treated with plasma to provide surfaces which are enriched in oxygen-containing groups and which provide accessible functional groups for attachment of biologically relevant compounds or cells. In additional embodiments, the present invention provides these plasma treated surfaces which are bound to a conjugate or cross-linker which can further bind to a biologically active compound or cell. Further, embodiments of the present invention provide methods for measuring the ability of the surface to bind to biologically active compounds.

Plastic surfaces treated with plasma are extensively used in commercial cell culture applications and research. Examples include “TCT” CellBIND® from Corning Incorporated Corning, N.Y., and Primaria® from BD Biosciences, Franklin Lakes, N.J. Even with these available surfaces, there is demand for additional surfaces that meet the needs of poorly adhering cells and cell lines. Stem cells, including human embryonic stem cells (hESCs) may not grow on these commercially available surfaces.

To address these problems, surface coatings have been developed to provide surfaces upon which culture-resistant cells will grow and thrive. Examples of these coatings include gelatin, collagen, fibronectin, laminin, synthetic peptide coatings such as poly-D-lysine and Matrigel®, a protein mixture secreted by mouse tumor cells, available from Beckton Dickinson, Franklin Lakes, N.J. In theory, these compounds may normally present in the extracellular matrix to which cells are attached in vivo. However, proteins used in these coating materials are largely animal derived, thus resulting in high cost, unpredictable lot to lot variability, poor shelf life and potential contamination of cell culture with animal-derived infective agents such as prions and viruses.

Non-animal derived surfaces have also been described. AlgiMatrix™ from Invitrogen, Carlsbad, Calif., is an alginate sponge. Ultra-Web™ is a synthetic nanofibrillar extracellular matrix sold by Surmodics, Eden Prarie, Minn., and Corning Incorporated, Corning, N.Y. Corning Incorporated, also sells polystyrene surfaces which have reactive functionalities available at the plastic surface such as Cell-BIND®, DNA-BIND® (N-oxysuccinimide), Sulfhydryl-BIND™ (maleimide), Carbo-BIND™ (hydrazide) and Universal-BIND™.

Providing activated surfaces is desirable in other fields as well. For example, it is desirable to provide a polymeric surface with active moieties that are available for binding with biologically active compounds for chemical, biochemical and biological assays and research. For example, ELISA methods may be made more sensitive if the material support for the assay binds to the reagents more efficiently. Enzyme activity assays, metabolite quantitation, nucleic acid quantitation, protein quantitation, cell viability, cell proliferation, cell function, cell differentiation and apoptosis studies are examples of assays that might be used with an activated polymeric surface.

In most applications it is desirable to achieve a high density of biologically active molecules at the surface of a polymer to provide a surface that is useful for applications related to the manipulation and handling of biologically active molecules. Surfaces with better ability to bind to biologically active molecules may be more efficient. They may magnify the measurable functional properties of analytes bound to the surface. That is, it may be preferable to provide a high density of functional groups on the polymer surface available for conjugation of biologically active compounds.

Treatment of a polymer with aggressive liquid reagents may result in a high density of functional groups at the surface of a polymer material, available for conjugation. For example, oxidation of polyethylene with chromium trioxide in sulfuric acid at 72° C. for 5 min and then in 70% nitric acid at 50° C. for 15 min produced carboxylic acid groups at a density of 33 pmol/mm² (James R. Rasmussen, Erwin R. Stedronsky, and George M. Whitesides, Introduction, Modification, and Characterization of Functional Groups on the Surface of Low-Density Polyethylene Film, J. Am. Chem. Sci., 99 (1977) 4736). Treatment of poly(methyl methacrylate) with 10M NaOH for 16 h at 40° C. produced carboxylic acid groups at a density of 6.6 nmol/mm² (Holmberg K., Hyden H., Methods of Immobilization of Proteins to Polymethylmethacrylate, Prep. Biochem. 15 (1985) 309)]. However, these methods require treatment of plastics for extended periods of time with concentrated corrosive solutions, which present a disposal problem and impose stringent safety guidelines and which may leave residues which may not be amenable to cell culture or other biological applications. Thus, wet chemistry processes may not be well suited for industrial applications. In addition, it may be that wet chemistry treatment is not restricted to the surface of the polymer, but likely may extend into the treated polymer at a depth. Thus, not all the functional groups imparted to the surface may be available for conjugation with often bulky biomolecules presented to the surface. Given these characteristics of wet chemical processes, plasma treatment and functional coatings remain major industrial processes for plastic surface modification and functionalization.

Plasma surface modification is attractive due to its low consumption rate of chemicals, fast processing speeds and low environmental impact. Although plasma treatment is very effective, it usually does not result in high densities of a specific functional group, but produces a spectrum of different functional groups. For example, oxygen plasma treatment of polystyrene produces carboxyl, carbonyl, hydroxyl, ester, ether and other oxygen containing groups, the ratio of which depends on the plasma treatment conditions. Plastic surfaces amenable to treatments according to embodiments of the present invention include polyacrylates, polymethylacrylates, polycarbonates, polystyrenes, polysulphones, polyhydroxy acids, polyanhydrides, polyolefins, polyorthoesters, polypropylenes, polyethylenes, polyphosphazenes, polyphosphates, polyesters, polyethers, nylons, cyclic polyolefin copolymers, acrylics, or mixtures thereof.

It is desirable to provide surfaces for cell culture, and for other applications, which are entirely synthetic, which do not leach undesirable materials, and which support adhesion and proliferation of cells. Cells adhere to surfaces through bonds made by proteins extending from the surfaces of the cells. That is, just as cells interact with extracellular matrix components through interactions with cell surface proteins, cells interact with the surfaces of cell culture containers through interactions with cell surface proteins. Some cells are particularly fastidious and difficult to culture. Some cells may prefer cell culture surfaces which have a higher number of active moieties upon which to bind, and some cells may prefer cell culture surfaces which have a lower number of active moieties for binding. That is, it is desirable to provide cell culture surfaces within particular ranges of concentrations of active moieties available for cells to bind. It is also desirable to provide multiple cell culture surfaces which provide a range of active moieties for binding, so that a customer can test their particular cell culture on a range of surfaces to allow them to choose the best environment for their particular cell culture needs.

It is also desirable to provide surfaces for the attachment of biologically relevant compounds such as peptides, proteins, carbohydrates, nucleic acid, lipids, polysaccarides, glycosaminoglycans, proteoglycans, hormones, extracellular matrix molecules, cell adhesion molecules, natural polymers, enzymes, antibodies, antigens, polynuceotides, growth factors, synthetic polymers, polylysine, drugs and other molecules or combinations or fragments of these. The binding of these compounds to a surface is relevant for the binding of cells to a surface as described above, and for use in biochemical tests and analyses, experiments and techniques, and in experiments and techniques which require tethering a compound to a surface to allow for further processing. It is also desirable to provide these surfaces where the surfaces are characterized in a way that allows users to gauge the relative surface binding characteristics of one surface versus another.

Methods for surface-treating polymeric surfaces have been described in, for example, U.S. Pat. No. 6,617,152, incorporated herein by reference in its entirety. That reference described treating polymeric materials including polyacrylates, polymethylacrylates, polycarbonates, polystyrenes, polysulphones, polyhydroxy acids, polyanhydrides, polyorthoesters, polypropylenes, polyphosphazenes, polyphosphates, polyesters, nylons or mixtures thereof. However, applicants have found that cyclic polyolefin copolymers, such as, but not limited to those sold under the trade name Topas® by TOPAS Advanced Polymers, Inc. Florence, Ky. and Zeonex®/Zeonor® sold by ZEON Inc., Tokyo, Japan, are useful as substrates for embodiments of the present invention. Cyclic polyolefin copolymer surfaces are relatively resistant to organic solvents and react positively to chemical and plasma treatments.

Treated polymer materials have commonly been characterized by XPS, X-ray Photoelectron Spectroscopy. Using this technique, the chemical make-up of the surface of a material can be characterized. Depending upon the angle at which the X-ray is directed at the surface when measurements are taken, which is known as the take-off angle measurements can be taken at different depths from the surface of the material. While this method can be used to identify the chemical species present at the surface, this method cannot measure the number of functional groups that are present at the surface, and which are available for attachment by a biologically relevant compound. For example, an XPS measurement of COOH carbons, taken at a take-off angle of 80° can yield information about the presence of COOH carbons up to a depth of between 5 and 10 nm below the surface. While this measurement is interesting, the reactive species may exist deep relative to the surface of the polymer. This deep reactive moiety may or may not be available to bind to a large bulky peptide or other biologically active compound presented to the surface of the polymer.

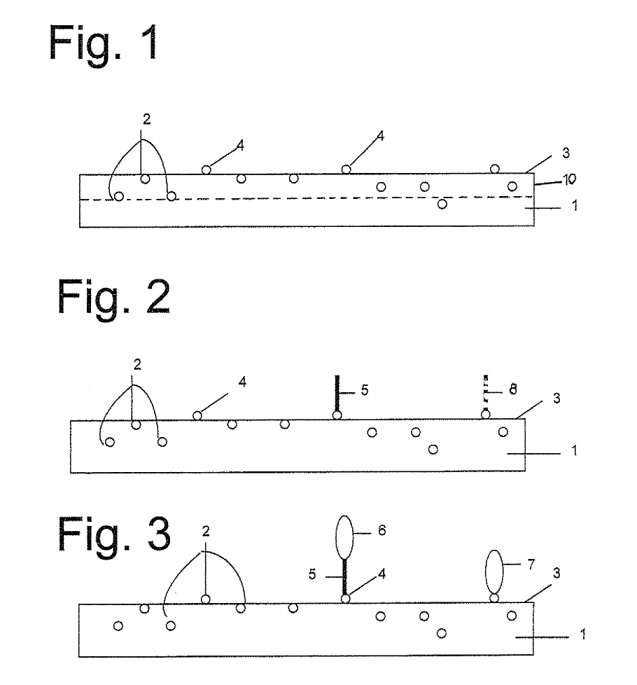

FIG. 1 illustrates a substrate or polymer 1 that has been treated, either by chemical treatment, plasma treatment or other treatment, where functional groups 2 have been introduced into the material. FIG. 1 illustrates that this treatment may result in the introduction of functional groups or reactive groups 2 into the material on the surface 3 of the polymer 1 and below the surface. Only a subset of these functional groups 4 are actually on the surface 3 of the polymer 1, although all of the functional groups may be counted using commonly used techniques such as XPS.

This modified polymer surface can be characterized by several known methods, including XPS, by measuring the contact angle of a droplet of water on the surface, by using dyes that interact with particular groups at the surface, by measuring the Zeta potential of the surface, by Fourier Transform Infrared Spectroscopy (FTIR), by atomic force microscopy (AFM), or by scanning electron microscopy (SEM). Each of these methods will characterize the number and character of functional groups at or near the surface of the polymer.

However, the number of functional groups at or near the surface of the polymer is not necessarily the most relevant information to describe that surface. For example, if the desired use of the treated surface is to attach a protein to the surface, the most relevant information about that surface may be how many proteins can attach to the modified surface, and not the number of polar groups that exist at or near the surface. For example, if a COC polymer treated with N₂O microwave plasma has a concentration of carboxyl groups at a depth of 100 Å as measured by XPS, that information may not be relevant to the availability of carboxyl groups to interact with a biomolecule at the polymer's surface. In addition, different surfaces, treated in different ways, will have different surface characteristics, leading to biologically active compound binding profiles that vary.

For example, the availability of surface functional groups for binding to biologically active compounds, organic compounds that have reactive groups such as peptides, proteins, carbohydrates, nucleic acid, lipids, polysaccarides, glycosaminoglycans, proteoglycans or combinations thereof, hormones, extracellular matrix molecules, cell adhesion molecules, natural polymers, enzymes, antibodies, antigens, polynuceotides, growth factors, synthetic polymers, polylysine, drugs and other molecules may be only indirectly described by XPS analysis. Instead of this physical measurement of chemical groups at a depth from a surface, a more relevant measurement of the ability of a surface to bind a biologically active compound is desirable.

In an embodiment of the method of the present invention, a substrate is provided which can bind a cell or a biologically active compound such as a peptide. In embodiments, the substrate is a cyclic polyolefin copolymer (COC) material that is treated by exposure to plasma to generate active groups at or near the surface of the polymer. Depending on the plasma treatment parameters, including the temperature, the gas used, the pressure, the volume, the shape of the surface, the humidity, the power of the energy source, and the flow rate of the plasma across the surface, more or fewer functional groups may be provided at or near the surface of the polymer. Depending upon the type of gas used for the treatment, different chemical groups may become introduced into the pre-surface layer of the polymer. The pre-surface layer of the polymer is the topmost layer of the polymer which can be accessed and altered by the treatment (see FIGS. 1, 10). Further, the polymer surface may be exposed to conjugate or cross-linker so that the functional groups on the surface of the polymer substrate form covalent bonds with the conjugate or cross-linker. The conjugated polymer surface can be further exposed to a biologically active compound so that the conjugate or conjugated polymer surface forms covalent bonds with the biological agent or, the biological agent can form bonds with the polymer surface so that no additional chemical structure remains between the biological agent and the polymer surface.

In embodiments, methods for measuring the ability of a surface to bind a biologically active compound are provided. For example, a surface is provided for characterization. This surface may be a polymer surface such as cyclic polyolefin copolymer, polystyrene, acrylic, or other surface. The surface may be treated, for example by exposure to plasma, UV light, heat, chemical or other treatment. The surface may be exposed to a conjugate so that the functional groups on the surface of the polymer form covalent bonds with the conjugate. The conjugate or cross-linking agent may be, for example, 1-ethyl-3-[3-dimethylaminopropyl]carbodiimide (EDC), N-hydroxysuccinimide (NHS), EDC/NHS mixture, 1-cyclohexyl-3-(2-morpholinoethyl)carbodiimide, dicyclohexyl carbodiimide, diisopropyl carbodiimide, N-ethyl-5-phenylisoxazolium-3′-sulfonate, N,N′-carbonyldiimidazole, 4-(p-azidosalicylamido)butylamine, 1,4-di-[3′-(2′-pyridyldithio)propionamido]butane, bismaleimidohexane, 1,5-difluoro-2,4-dinitrobenzene, 4,4′-difluoro-3,3′-dinitrophenylsulfone, N,N′-ethylene-bis(iodoacetamide), N,N′-hexamethylene-bis(iodoacetamide), N,N′-undecamethylene-bis(iodoacetamide), α,α′-diiodo-p-xylene sulfonic acid, tris(2-chloroethyl)amine, glutaraldehyde, formaldehyde, dithio bis(succinimidyl propionate), 3,3′-dithio bis(sulfosuccinimidyl propionate), disuccinimidyl suberate, bis(sulfosuccinimidyl)suberate, disuccinimidyl tartarate, bis[2-(succinimidyloxycarbonyloxy)ethyl]sulfone, ethylene glycol bis(succinimidyl succinate), disuccinimidyl glutarate, N,N′-disuccinimidyl carbonate, dimethyl adipimidate, dimethyl pimelimidate, dimethyl suberimidate, dimethyl 3,3′-dithiobispropionimidate, N-hydroxysuccinimidyl-4-azidosalicylic acid, sulfosuccinimidyl-2-(p-azidosalicylamido)ethyl-1,3′-dithiopropionate, N-hydroxysuccinimidyl-4-azidobenzoate, N-succinimidyl-6-(4′-azido-2′-nitrophenylamino)hexanoate, N-5-azido-2-nitrobenzoyloxysuccinimide, sulfosuccinimidyl-2-(p-(m-azido-o-nitrobenzamido)-ethyl-1,3′-dithiopropionate, N-succinimidyl(4-azidophenyl)-1,3′-dithiopropionate, sulfosuccinimidyl-4-(p-azidophenyl)butyrate, sulfosuccinimidyl-2-(7-azido-5-methyl coumarin-3-acetamide)ethyl-1,3′-dithiopropionate, sulfosuccinimidyl-7-azido-4-methylcoumarin-3-acetate, p-nitrophenyl diazopyruvate, N-succinimidyl 3-(2-pyridyldithio)propionate, 4-succinimidyloxycarbonyl-α-methyl-α-(2-pyridyldithio)toluene, succinimidyl 4-(N-maleimidomethyl)-cyclohexane-1-carboxylate, m-maleimidobenzoyl-N-hydroxysuccinimide ester, N-succinimidyl(4-iodoacetyl)-aminobenzoate, succinimidyl 4-(p-maleimidophenyl)butyrate, succinimidyl 6-[6-(((iodoacetyl)amino)-hexanoyl)amino]hexanoate, succinimidyl 6-[(iodoacetyl)-amino]hexanoate, succinimidyl 6-((((4-iodoacetyl)amino)methyl-cyclohexane-1-carbonyl)amino)hexanoate, succinimidyl 4-((iodoacetyl)amino)methyl)-cyclohexane-1-carboxylate, p-nitrophenyl iodoacetate, 1-(p-azidosalicylamido)-4-(iodoacetamido)butane, N-[4-(p-azidosalicylamido)butyl]-3′-(2′-pyridyldithio)propionamide, 1,4-butanediol diglycidyl ether, adipic acid dihydrazide, carbohydrazide, p-nitrophenyl-2-diazo-3,3,3-trifluoropropionate, p-azidobenzoyl hydrazide, benzophenone-4-iodoacetamide, benzophenone-4-maleimide, 4-(4-N-maleimidophenyl)butyric acid hydrazide hydrochloride), 4-(N-maleimidomethyl)cyclohexane-1-carboxyl-hydrazide hydrochloride, and 3-(2-pyridyldithio)propionyl hydrazide. (See Goddard et al, Prog. Polym. Sci. 32 (2007) 698-725 at p. 706).

In embodiments, the surface with a bound conjugate may then be exposed to a relevant biologically active agent so that the biologically active agent is immobilized on the surface of the polymer. The biologically active agent may be labeled. For example, the biologically active agent may be labeled with a fluorophore, a chemical tag in a molecule which will absorb energy of a specific wavelength and re-emit energy at a different (but equally specific) wavelength. Fluorescein isothiocyanate, a reactive derivative of fluorescein, has been one of the most common fluorophores chemically attached to other, non-fluorescent molecules to create new and fluorescent molecules for a variety of applications. Other historically common fluorophores are derivatives of rhodamine, coumarin, cyanine, (CyDyes), pyrene, naphthalene, bimane, pyridyloxazole, benzoxadiazole, dapoxyl, diazoalkane, BODIPY fluorophores, Alexa Fluor dyes, and other fluorescent markers. The biologically active agent may be labeled with, for example fluorescent markers such as rhodamine (which identifies carboxylic acid) or fluorescein (quaternary ammonium compounds), or other markers such as toluidine blue O (carboxylic acid), acid orange 7 (primary amine), picric acid (primary amine), thionine acetate (carboxylic acid), acriflavin (carboxylic acid), ethidiumbromide (carboxylic acid), 1,1-diphenyl-2-picryllhydrazyl (free radicals), amine functionalized BODIPY (carboxylic acid), dansyl cadaverine (carboxylic acid), succinimide ester functionalized BODIPY (primary amine), fluorescamine (primary amine), fluorescein-5-isothiocyanate (primary amine), dansyl chloride (hydroxyl), labeled streptavidin (biotin), Dragendorff reagent (polyethylene glycol), Schiff's reagent, Ellman's reagent, bromocresol green, or ninhydrin. Radiolabels such as ³²P, ³⁵S, T, or ¹⁴C, isotopic labels such as D, ¹⁵N, ¹³C or other markers may also be used. After appropriately rinsing the surface, the amount of label bound to the surface may be determined using fluorescence readers, or other appropriate methods.

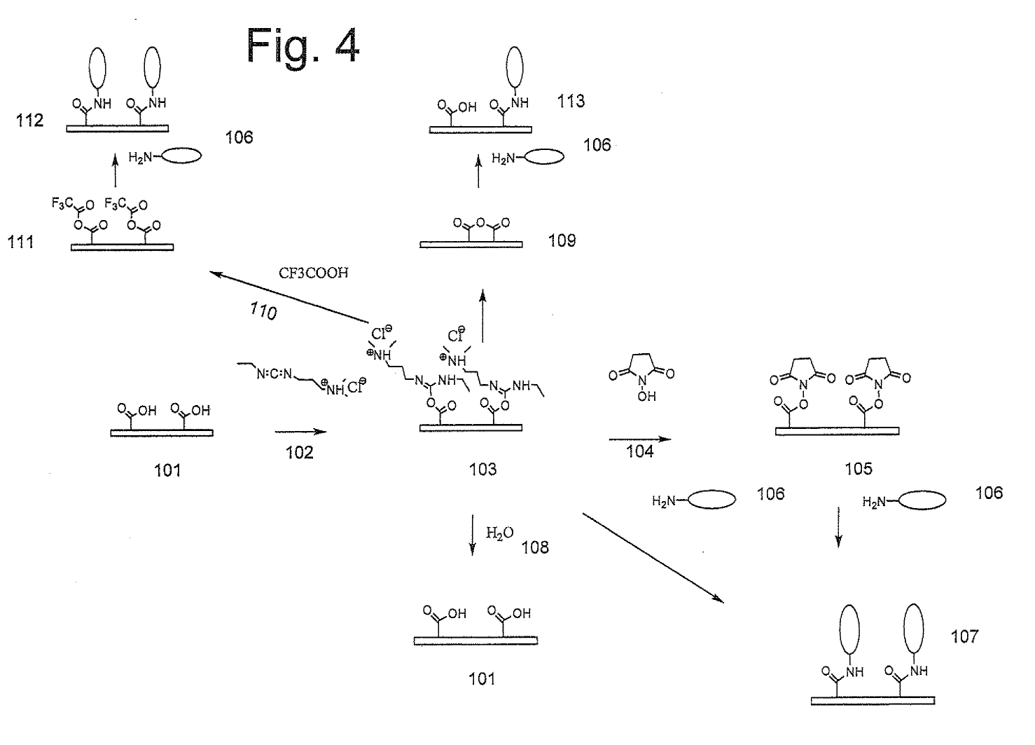

FIG. 2 illustrates an embodiment of the present invention showing a polymer 1 that has been treated to create functional groups 4 at the surface 3 of the polymer 1. This treated polymer surface has been further exposed to a conjugate or cross-linking agent such as 1-ethyl-3-[dimethylaminopropyl]carbodiimide (which may be in the form of carbodiimide hydrochloride) (EDC) or a combination of EDC and N-hydroxysuccinimide (NHS). This reaction forms activated groups at the surface of the polymer that are available for binding to biologically active compounds which have free primary amine groups, such as peptides and proteins. This chemistry is illustrated in FIG. 4.

FIG. 3 illustrates an embodiment of the present invention showing a polymer 1 that has been treated to create functional groups 4 at the surface 3 of the polymer 1, where the treated polymer 1 has been further exposed to a conjugate or cross-linking agent 5, and where the conjugated treated polymer has been further exposed to a biologically active agent which has been labeled 6. This labeled biologically active agent is, for example, a peptide labeled with tetramethylrhodamine (TAMRA), a fluorescent marker. After a washing step, the amount of labeled protein present on the surface on the polymer can be assessed. FIG. 3 also illustrates that the conjugate or cross-linking agent may be present (as shown where the biologically active agent 6 is linked to the conjugate 5 which is linked to the functional group 4 at the surface of the substrate 1) or may not be present (as shown where the biologically active agent 7 is linked directly to the functional group 4 at the surface of the substrate 1) upon the completion of the reaction. For example, the cross-linking agent may be a zero length cross-linker. This is shown, for example, in FIG. 4. 107 which shows that when the conjugate or cross-linking agent is EDC/NHS, the conjugate or cross-linking agent is not present upon completion of the reaction. In some embodiments, the conjugate 5 is not necessarily present after exposure to the biologically active agent.

In additional embodiments, a linker may be present, as shown, for example in FIG. 2 as linker 8. In this embodiment, the linker may be, for example polyethylene glycol (PEG) or polyethylene oxide (PEO) or similar linker compounds. These linker compounds serve to elevate the biologically active agent 6, which may be bound to the linker, away from the surface of the substrate. This may be important to allow for the binding of bulky biologically active agents or cells to the surface.

Comparing FIG. 1 with FIG. 3, it can be seen that a polymer surface which has been treated may have a significant number of functional groups that would be detected by using methods such as XPS, but measuring these groups is not necessarily related to the number of functional groups that are available at the surface of the treated polymer which are available for binding to a biologically active compound. It is the degree to which the surface has functional groups that are available for binding to a biologically active compound which is the true feature of the surface that is of interest to scientists who might need surfaces with binding characteristics that fall within a range of binding availability to meet their specific needs.

For example, in the field of cell culture, some cells may attach to a polymeric surface if that surface exhibits a certain range of binding density, but not if the polymeric surface is outside that range. Some populations of cells may grow more productively on a polymeric surface with a certain binding affinity, while other populations of cells may respond negatively to that same surface characteristic. Especially in the field of cell culture, but in other fields as well, it is very difficult to compare the binding characteristics of one surface to another surface, because the parameters that lead to successful cell growth are complex.

In an embodiment of the present invention, a method for assessing the number of accessible functional groups on the surface of a polymer available for cell binding or binding of a biologically active agent is provided. A polymer surface is provided. This surface can be, for example, a plasma-treated cyclic polyolefin copolymer surface having a number of functional groups on its surface to be determined. The polymer surface can be exposed to, for example, a conjugate or cross-linking agent such as 1-ethyl-3-[dimethylaminopropyl]carbodiimide (EDC)), or a combination of EDC and N-hydroxysuccinimide (NHS) so that the carboxyl groups on the surface of the plasma-treated cyclic polyolefin copolymer form a temporary covalent bond with the 1-ethyl-3-[dimethylaminopropyl]carbodiimide or N-hydroxysuccinimide. This treated polymer can then be exposed to a labeled biological agent such as, for example, a tetramethylrhodamine-labeled peptide, so that the labeled biological agent is immobilized on the polymer surface. The amount of bound labeled biological agent can then be determined.

Using this method, the number of accessible functional groups on the surface of the polymer that are available for cell binding or binding of a biologically active agent can be assessed. Using this method, a user may be better able to identify a surface which might work well for that user's application, using a method directly related to the level of binding that is possible on a surface of a polymer substrate.

The degree to which a treated surface, exposed to a conjugate or cross-linking agent can bind a biologically active compound such as a peptide or protein described above, can be measured by measuring the amount of label bound to the surface. A rating can then be assigned to the surface based on the amount of binding measured using an embodiment of the methods of the present invention. The rating can be, for example, a Conjugation Density Rating, or a Binding Rating or a Surface Binding Rating. Those of skill in the art will recognize that the name given to the rating system can be changed without deviating from the spirit of the invention.

The amount of label bound to the surface, and the rating applied to the system based on a measurement of label bound to the surface, will depend upon the surface itself, the nature of treatments applied to the surface, the type, quantity and nature of a conjugate or cross-linking agent applied to the surface, and the type, quantity and nature of the biologically active compound applied to the conjugated or cross-linked surface, and conditions under with they are applied.

For example, a surface which might be COC or polystyrene (PS) may be treated with plasma and/or treated with a conjugate compound. A desired labeled biologically active compound may then be applied to the surface, for example a peptide protein or a nucleic acid. After appropriate washing the binding ability of the surface(s) may then be measured. The same surface may have a different rating for the protein vs the nucleic acid binding. However, the rating information is valuable based on a standardized method for treating and analyzing the surface to provide a rating. A standard rating system that is based on relevant surface characteristics may allow a purchaser of treated surfaces to more easily compare one surface to another, making the process of deciding which surface to purchase more efficient.

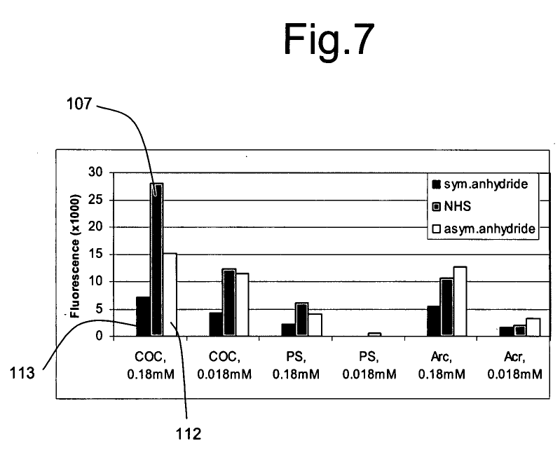

FIG. 4 illustrates an example chemistry for producing a polymer surface which is reacted with conjugates or cross-linking compounds to bind with biologically active compounds having primary amine groups. FIG. 4 illustrates a surface with carboxylic acid groups 101, such as a plasma treated polymer surface. This surface can then be exposed to a conjugate or cross-linker agents such as, but not limited to, 1-ethyl-3-[dimethylaminopropyl]carbodiimide (EDC) 102. EDC may be a HCL salt or may be provided in another form. This exposure may create an unstable reactive O-acylisourea 103 which can be treated with a biologically active compound having a primary amine group 106 in the presence or absence of acyl transfer agents such as, but not limited to, N-hydroxysuccinimide, N-hydroxysulfosuccinimide sodium salt, p-nitrophenol, pentafluorophenol, 1-hydroxybenzotriazole, 1-hydroxy-7-azabenzotriazole, ethyl 1-hydroxy-1H-1,2,3-triazole-4-carboxylate, and methyl imidazole. In some cases it would be beneficial to react the O-acylisourea ester with N-hydroxysuccinimide (NHS) 104 and then with a biologically active compound to form a substrate having a biologically active compound 107 bound to the surface through a stable amide bond. This structure may be the same as that shown as 112. Or, 107 and 112 may have different chemistries of biomolecules bound to the surfaces (see FIG. 7). Where the asymmetrical anhydride bar corresponds with 112 of FIG. 4, the NHS bar of FIG. 7 corresponds with 107 of FIG. 4, and the symmetrical anhydride chemistry correlates with 114 of FIG. 4. In the absence of NHS, the unstable reactive O-acylisourea may form stable amide bonds between the substrate and the biologically active compound 113 through the formation of symmetric anhydride 109. Or, in step 110, unstable reactive O-acylisourea may form asymmetric anhydride 111 in the presence of acid such as trifluoroacetic acid. Upon treatment with the biologically active compound having a primary amine group 106, a stable bond can be formed between the substrate and the biomolecule 112. The biologically active compound may be labeled. Reactive N-hydroxysuccinimide ester is easily hydrolysable in the presence of moisture, thus it may be beneficial to perform the conjugation steps in dry organic solvent to increase the efficiency of the conjugation steps. If reactive O-acylisourea is exposed to moisture 108, the carboxyl group is regenerated 101.

Embodiments of the present invention are further illustrated in the following examples.

### EXAMPLES

### Example 1

### Chemical Conjugation on a Polymer Surface

A treated polymer, a cyclic polyolefin copolymer (COC), treated with microwave plasma to generate carboxylic acid groups on its surface was provided 101. The surface was exposed to microwave plasma in a Plasma-Preen II 973 microwave plasma system using humid air as a processing gas. The duration of the treatment was 30 seconds at a power setting of 50%, corresponding to approximately 650 W. The COC sample was placed in the Plasma-preen II 973 microwave in a glass bell jar reactor that is then evacuated with an oil vacuum pump and purged with processing gas during plasma treatment. Pressure inside the plasma reactor was approximately 10⁻² to 10⁻³ Torr. The temperature within the plasma reactor was room temperature initially (approximately 25° C.), and rose during treatment to approximately 30° C.

This treated COC polymer surface was then treated with 0.1 M 1-ethyl-3-[dimethylaminopropyl]carbodiimide (EDC) 102 and 0.05M N-hydroxysuccinimide (NHS) 104 in dimethylformamide (DMF) for 1.5 hours to convert carboxyl groups on the polymer surface to N-hydroxysuccinimide ester groups 105, via an unstable reactive O-acylisourea, shown as 103 in FIG. 4. This NHS-ester polymer was then exposed to a fluorescently labeled biologically active compound having a primary amine group 106 for 1.5 hours. The labeled compound 106 was a mixture of fluorescently labeled/unlabeled peptide amides (RGGSDPIYK—NH₂/TAMRA-GRGDSPIIK—NH₂) in a ratio of 99:1 in 25 mM phosphate buffer, pH 7.4. (SEQ ID NO 1: RGGSDPIYK) (SEQ ID NO 2: GRGDSPIIK).

The mixture of labeled/unlabeled peptide was used to increase distances between fluorescently labeled molecules conjugated to the surface. When fluorescence molecules are in close proximity, quenching of the fluorescent signal occurs, which results in non-linear dependence of fluorescent signal vs. amount of fluorophore. This is a common phenomenon associated with fluorescence method.

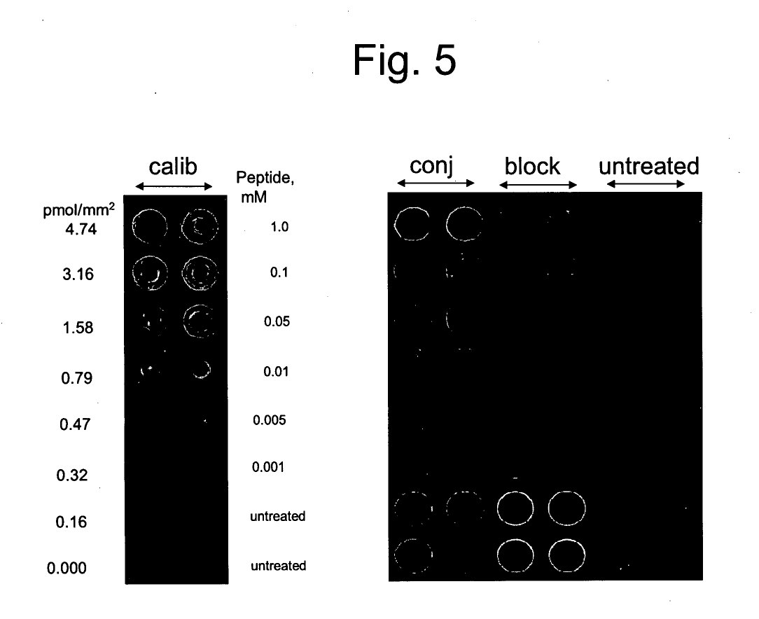

FIG. 5 illustrates peptide binding of a treated polymer, with a calibration curve compared to conjugated, negative control (blocked) and untreated surfaces. In a negative control, NHS ester was reacted with ethanolamine or N-(3-aminopropyl)morpholine (1-3M solutions at pH 7.5 to 8.0) for 1.5 hours prior to exposure to a mixture of fluorescently labeled/unlabeled peptide amides (H-RGGSDPIYK(SEQ ID NO 1)-NH₂/TAMRA-GRGDSPIIK(SEQ ID NO 2)-NH₂, 99:1) in 25 mM phosphate buffer, pH 7.4. The surface is then washed with deionized water and dried. Negative control experiments were performed to determine the amount of peptide adsorbed onto the surface after washing. Thus, to obtain a true fluorescence of the peptide conjugated to the surface, the fluorescence of blocked wells should be subtracted from the fluorescence of conjugated wells.

Labeled peptide conjugation was performed in parallel with the calibration tests to estimate the density of peptides bound to the surface. The calibration curve was generated by dispensing a known amount of fluorescently labeled/unlabeled peptide mixture (RGGSDPIYK(SEQ ID NO 1)-NH₂/Rhod-GRGDSPIIK(SEQ ID NO 2)-NH₂, 99:1) in DI water into wells and evaporating water under vacuum. The calibration curve measures fluorescence at total peptide concentrations of 0, 0.16, 0.32, 0.47, 0.79, 1.58, 3.16 and 4.74 pmol/mm².

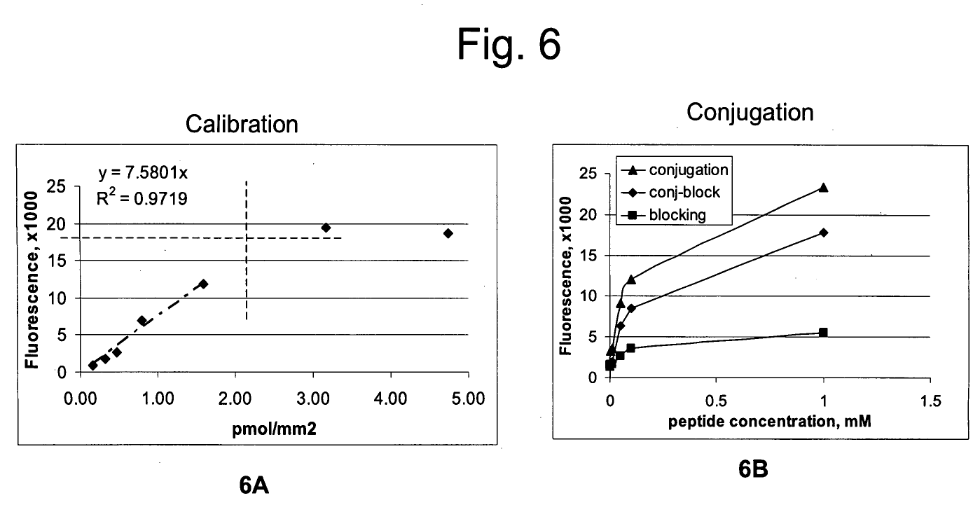

Conjugation of labeled/unlabeled peptide mixture to the activated surface was performed in 25 mM phosphate buffer (pH 7.4) using seven peptide solutions of diminishing concentrations: 1 mM, 0.1 mM, 0.05 mM, 0.01 mM, 0.005 mM, and 0.001 mM. The experiment was designed this way to monitor an increase in peptide density (fluorescence intensity) at the surface as the concentration of peptide in conjugation buffer was increased. These results are shown in FIG. 6B. By increasing peptide concentration in the conjugation buffer, efficiency of the peptide conjugation step is increased, which should translate into higher fluorescence intensity. If, for example, decrease in fluorescence had been observed upon an increase in peptide concentration in conjugation buffer, this would indicate fluorescence quenching. In that case, the ratio of unlabeled/labeled peptide would need to increase to space fluorescently labeled peptides farther from each other on the surface. By comparing fluorescence of the conjugated peptides to that of known concentrations of peptide in the calibration curve, the density of conjugated peptides was estimated. It may be beneficial to conjugate the peptide to the surface in the calibration wells of the microplate using the same conjugation strategy to improve surface coverage uniformity and ensure linearity of the calibration curve.

Fluorescence was measured with a TECAN microarray scanner (×1000). FIGS. 6A and 6B illustrate the measured fluorescence of the calibration curve and the conjugation experiments, as shown in FIG. 5. FIG. 6A shows a plot of fluorescence vs peptide density generated from the calibration curve. FIG. 6A illustrates a concern with fluorescence measurement of peptides under these circumstances. At concentrations of up to 1.75 pmol/mm², fluorescence measurements were approximately linear, as would be expected. However, between 3 and 5 pmol/mm², fluorescence measurements flattened out, and even declined. This is due to fluorescence quenching, as a result of non uniform surface coverage with peptide in calibration wells. Adjusting the volume of peptide solutions and/or addition of solvents may help to remediate the problem. This is an especially useful strategy from the perspective of conjugation of peptide to the surface for calibration.

FIG. 6B shows a plot of fluorescence intensities for conjugated and blocked wells, as well as subtraction of one from another (conj-block) representing a true fluorescence of conjugated peptide. Thus, although peptide densities up to 2.3 pmol/mm² were achieved in described experiments, to those skillful in the art it is clear that any peptide density from 0-2.3 pmol/mm² could be easily achieved by adjusting conditions under which the conjugation or surface activation is performed. For example, based on the calibration and conjugation data presented, one could use 0.015 mM solution of peptide to achieve a peptide density of 0.5 pmol/mm², while 0.4 mM solution of peptide would result in a peptide density of 1.5 pmol/mm² under the same conditions.

### Example 2

### Peptide Conjugation to Different Surfaces

FIG. 7 illustrates peptide conjugation on three different surfaces, cyclic polyolefin copolymer (COC), polystyrene (PS) and acrylic (Acr). Each surface was exposed to microwave plasma in a Plasma-Preen II 973 microwave plasma system using humid air as a processing gas. The duration of the treatment was 30 seconds at a power setting of 50%, corresponding to approximately 650 W. Each sample was placed in the Plasma-preen II 973 microwave in a glass bell jar reactor is then evacuated with an oil vacuum pump and purged with processing gas during plasma treatment. Pressure inside the plasma reactor was approximately 10⁻²-10⁻³ Torr. The temperature within the plasma reactor was room temperature initially (approximately 25° C.), and rose during treatment to approximately 30° C.

After microwave plasma treatment, the carboxylic acid groups at the surface were activated using 0.4 M EDC (1-ethyl-3-[3-dimethylaminopropyl]carbodiimide) and 0.1 M NHS (N-hydroxysuccinimide) in water for 10 minutes. Then the solutions were replaced with fluorescently labeled peptides in pH 7.4, mM phosphate buffer solution at a concentration of 0.018 mM and 0.18 mM. Fluorescently labeled peptide was left to react with the treated surfaces for 30 minutes then the conjugation solutions were aspirated and the surfaces were washed with phosphate buffer having 1% SDS (sodium dodecyl sulfate), and then washed again with Di water. The fluorescent intensities were measured using a microarray scanner.

FIG. 7 shows that the COC surface treated with EDC/NHS provided a surface which exhibited more than twice the binding capacity of similarly treated polystyrene or acrylic surfaces. This result is surprising. The COC surface, treated with EDC/NHS results in a surface that allows immobilization of biomolecules at high densities compared to other plasma treated plastics.

FIG. 7 also illustrates that surfaces go through different reactions, for example the formation of symmetric or asymmetric anhydride intermediates before binding with a biologically active compound (shown as 109 and 111 in FIG. 4) form surfaces which do not bind biologically active compounds at the same density as the EDC/NHS treated surfaces.

### Example 4

### Cell Adhesion Assay

To evaluate the efficiency of peptide conjugation using the conjugation methods shown in FIG. 4, for cell adhesion, an HT-1080 cell adhesion assay was performed. Some of these surfaces were further processed according to the EDC/NHS chemistry shown in FIG. 4.

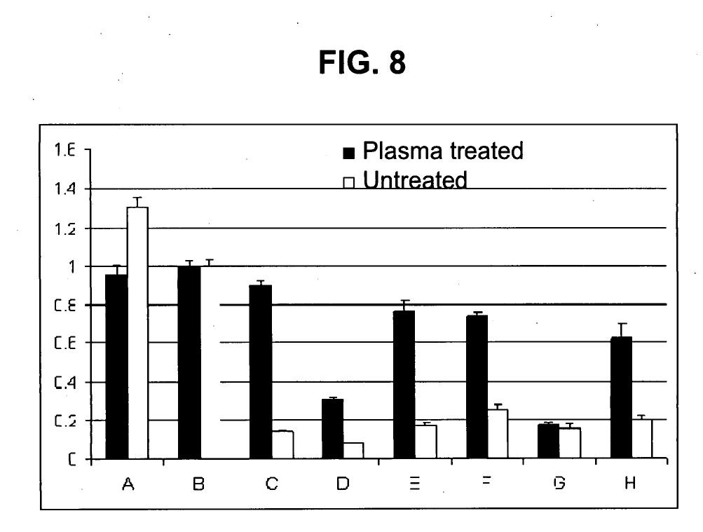

Plasma-treated (black bars) and untreated (white bars) cyclic polyolefin copolymer surfaces (Topas®), in the form of 96 well plates, were prepared. For the control data shown as “A” (fibronectin) and “B” (laminin) in FIG. 8, laminin and figronectin control wells were coated for 1 hour at room temperature with the respective protein (5 μg/mL, Sigma-Aldrich, St. Louis, Mo.). All wells were blocked with 1% BSA in PBS for 1 hour at 37° C. Wells were washed briefly with PBS before incubation with 0.1% BSA in Iscove's Modified Dulbecco's Media (IMDM) prior to cell seeding.

HT-1080 human fibrosarcoma cells (ATCC number: CCL-121) were seeded on control plates and peptide-conjugated plates at a density of 30,000 cells/well. Cell adhesion was allowed to take place for 1 hour at in IMDM (Lonza, Basel, Switzerland) with 10% FBS (Lonza) standard cell culture conditions. The media was aspirated from the wells and adherent cells were fixed and stained in 50 μL of 0.2% crystal violet in 20% methanol for 5 minutes at room temperature. Crystal violet dye was aspirated from the wells, and all surfaces were washed 3 times with water. Cellular absorption of crystal violet was quantified through addition of 1% SDS in H₂O for 5 minutes prior to absorbance measurement at 570 nm.

FIG. 8 is a graph showing HT-1080 cell binding on various surfaces prepared according to embodiments of the present invention. The data presented in FIG. 8 has been normalized to laminin-coated plasma treated and plasma-untreated cyclic polyolefin copolymer (Topas®) surfaces. Laminin-coated plasma treated and plasma-untreated cyclic polyolefin copolymer (Topas®) surfaces are shown as B in FIG. 8.

In FIG. 8, controls A and B show cell binding to fibronectin-coated surfaces and laminin-coated cyclic polyolefin copolymer surfaces with and without plasma treatment, in the absence of NHS/EDC chemistry and peptide binding. Control C shows cell binding on plasma treated (black bar) and untreated cyclic polyolefin copolymer surface, in the absence of further conjugation or the application of peptides. Control surface D shows cell binding on plasma treated (black bar) and untreated (white bar) cyclic polyolefin copolymer surfaces which have been blocked with bovine serum albumin (BSA).

Bars E, F and H show cell binding to surfaces (plasma treated in the black bars and untreated in the white bars) which have been conjugated with peptides (conjugation consists of first NHS/EDC activation followed by covalent immobilization of the peptide to the surface, as shown in FIG. 4). The surfaces shown in E were treated with BSP peptide (Ac—KGGNGEPRGDTYRAY—NH₂ (SEQ ID NO 3), a peptide known to enhance cell binding, or a cell-binding peptide. Cell growth data shown in F were obtained after conjugating plasma treated and untreated surfaces with a poly(ethylene oxide) (PEO) linker having four repeat and BSP peptide (NH₂—PEO₄—NGEPRGDTYRAY—NH₂) (SEQ ID NO 4), a peptide known to enhance cell binding or a cell-binding peptide. Cell growth data shown in H were obtained after conjugating plasma treated and untreated surfaces with vitronectin peptide (Ac—KGGPQVTRGDVFTMP—NH₂ (SEQ ID NO 5), another peptide known to enhance cell binding or a cell-binding peptide. G is an ethanolamine treated negative control. An EDC/NHS treated, plasma treated COC material was treated with a 1 M, pH 8.1 solution of ethanolamine to inactivate the NHS esters by immobilizing ethanolamine to the surface. Peptide solutions were then added and the sample processed normally. This negative control treatment ensures that no peptides can be immobilized on the surface.

FIG. 8 shows that plasma treatment of cell culture surfaces is important for cell binding, with (see E, F and H) and without peptide binding (see C). Plasma treatment of cell culture surfaces is also important for peptide immobilization (see E, F and H, in view of G, the negative control for peptide binding). Binding a known cell-adhesive peptide to the surface, with and without linker structures, enhances cell binding (see E, F and H). In addition, FIG. 8 shows that the cell binding response is from peptides that are covalently attached to the surface, not from peptides that are non-specifically bound to the surface (see E and G, black bars).

Examples of substrates that can be treated by the method disclosed herein include but are not limited to: flasks, dishes, flat plates, sheets, well plates, bottles, roller bottles, containers, pipettes, pipette tips, tubes, sheets, medical devices, filter devices, beads, membranes, slides, and medical implants. These items are typically formed by commonly practiced techniques such as injection molding, extrusion with or without end capping, blow molding, injection blow molding, etc. For example, beads including microbeads or microcarrier beads can be made by micro-emulsion polymerization of thermoplastics. They can be made by using industrial techniques such as jet cutting, electrostatic generator, resonance nozzle or rotative or spin disk.

Embodiments of the invention are targeted for cell adhesion, attachment, and growth as well as conjugation or binding of a number of biologically or chemically active molecules including but not limited to: peptides, proteins, carbohydrates, nucleic acids, lipids, polysaccarides, glycosaminoglycans proteoglycans or combinations thereof, hormones, extracellular matrix molecules, cell adhesion molecules, natural polymers, enzymes, antibodies, antigens, polynuceotides, growth factors, synthetic polymers, polylysine, drugs, and other molecules. Any cell type known to one of skill in the art may be attached and grown on the treated substrates of the present invention. Examples of cell types which can be used include nerve cells, epithelial cells, mesenchymal cells, stem cells, fibroblast cells, hepatocytes and other cell types.

The foregoing description of the specific embodiments reveals the general nature of the invention that others can, by applying knowledge within the skill of the art, readily modify and/or adapt for various applications such specific embodiments, without undue experimentation and without departing from the general concept of the present invention. Such adaptations and modifications, therefore, are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of skill in the art.

We claim:

1. A method for making a polymer substrate having a working surface upon which biologically active compounds can bind comprising:
providing a cyclic polyolefin copolymer;
treating the cyclic polyolefin copolymer with plasma to provide functional groups on a surface of the polymer substrate;
exposing the plasma-treated copolymer to a conjugate so that the functional groups on the surface of the polymer substrate form covalent bonds with the conjugate;
exposing the polymer surface covalently bound to the conjugate to a biologically active compound so that the biologically active compound is immobilized on the polymer surface.

2. The method of claim 1 wherein the conjugate comprises 1 ethyl-3-(3-dimethylaminopropyl)carbodiimide (EDC) or a combination of EDC and N-hydroxysuccinimide (NHS).

3. The method of claim 1 wherein the biologically active compound is selected from the group consisting of peptides, proteins, carbohydrates, nucleic acids, lipids, polysaccarides, glycosaminoglycans, proteoglycans, extracellular matrix molecules, cell adhesion molecules, or combinations or fragments thereof.

4. The method of claim 3 wherein the biologically active compound is a peptide.

5. The method of claim 1 wherein the cyclic polyolefin copolymer is treated with microwave plasma.

6. The method of claim 4 wherein the polymer substrate has a peptide binding density greater than 0.5 pmol/mm².

7. The method of claim 4 wherein the polymer substrate has a peptide binding density greater than 1.5 pmol/mm².

8. The method of claim 1 wherein the polymer substrate provides at least a portion of a flask, a dish, a flat plate, a sheet, a well plate, a bottle, a roller bottle, a container, a pipette, a pipette tip, a tube, a bead, a medical device, a filter device, a membrane or a slide.

9. A polymer substrate made by the method of claim 1.

10. A method for making a polymer substrate having a working surface upon which cells can bind comprising:
providing a cyclic polyolefin copolymer;
treating the cyclic polyolefin copolymer with plasma to provide functional groups on a surface of the polymer substrate;
exposing the polymer surface to a conjugate so that the functional groups on the surface of the polymer substrate form covalent bonds with the conjugate;
exposing the polymer surface with covalently bound conjugate to a cell-binding peptide so that the cell binding peptide becomes immobilized on the polymer surface.

11. The method of claim 10 wherein the conjugate comprises 1-ethyl-3-(3-dimethylaminopropyl)carbodiimide (EDC) or a combination of EDC and N-hydroxysuccinimide (NHS).

12. The method of claim 10 wherein the cell binding peptide comprises KGGNGEPRGDTYRAY (SEQ ID NO 3), NGEPRGDTYRAY (SEQ ID NO 4), KGGPQVTRGDVFTMP (SEQ ID NO 5), or a combination of these.

13. The method of claim 10 wherein the cell-binding peptide is immobilized on the polymer surface via a linker.

14. The method of claim 13 wherein the linker comprises PEG or PEO.

15. The method of claim 10 wherein the cyclic polyolefin copolymer is treated with microwave plasma.

16. The method of claim 10 wherein the polymer substrate provides at least a portion of a flask, a dish, a flat plate, a sheet, a well plate, a bottle, a roller bottle, a container, a pipette, a pipette tip, a tube, a bead, a medical device, a filter device, a membrane or a slide.

17. A method for assessing the number of accessible functional groups on the surface of a polymer available for binding comprising;
providing a plasma-treated cyclic polyolefin copolymer surface having a number of functional groups on its surface to be determined;
exposing the plasma-treated cyclic polyolefin copolymer surface to 1-ethyl-3-(3-dimethylaminopropyl)carbodiimide (EDC) and N-hydroxysuccinimide (NHS);
exposing the plasma-treated cyclic polyolefin copolymer surface to labeled biological agent or a mixture or labeled and unlabeled biological agents so that biological agent is immobilized to the surface, and;
determining the amount of label indirectly bound to the surface.

18. The method of claim 17 wherein the biological agent is a labeled polypeptide.

19. A polymer substrate having a working surface upon which cells can bind comprising a cyclic polyolefin copolymer substrate having plasma-treatment-induced functional groups conjugated to cell-binding peptides.

20. The polymeric substrate of claim 19 wherein the cell binding peptide comprises KGGNGEPRGDTYRAY (SEQ ID NO 3), NGEPRGDTYRAY (SEQ ID NO 4), KGGPQVTRGDVFTMP (SEQ ID NO 5), or a combination of these.

21. A method for assigning a rating to a surface comprising:
providing a treated polymer surface having a number of functional groups on its surface to be determined;
exposing the polymer surface to conjugate so that the functional groups on the surface of the polymer form covalent bonds with the conjugate;
exposing the polymer surface covalently bound to the conjugate to labeled biological agent or a mixture or labeled and unlabeled biological agents so that the labeled biological agent becomes immobilized to the polymer surface;
determining the amount of label immobilized to the polymer surface;
assigning a rating to the polymer surface based on the amount of label immobilized to the surface.


SEQUENCE LISTING (5 sequences)

SEQ ID NO: 1 - protein, 9 aa, Unknown
  synthetic peptide
RGGSDPIYK

SEQ ID NO: 2 - protein, 9 aa, Unknown
  synthetic peptide
GRGDSPIIK

SEQ ID NO: 3 - protein, 15 aa, Unknown
  synthetic peptide
KGGNGEPRGDTYRAY

SEQ ID NO: 4 - protein, 12 aa, Unknown
  synthetic peptide
NGEPRGDTYRAY

SEQ ID NO: 5 - protein, 15 aa, Unknown
  synthetic peptide
KGGPQVTRGDVFTMP